(19) United States
(12) Patent Application Publication
(10) Pub. No.: US 2026/0335141 A1
(43) Pub. Date: Oct. 29, 2026

(54) PETAL AND LEAF -SHAPED HERBAL INFUSED ANTIMICROBIAL ADHESIVE DRESSINGS AND APPLICATION

(71) Applicant: HOLISTIKIT, INC, Napa, CA (US)

(72) Inventor: Kirsty Shelton, Napa, CA (US)

(21) Appl. No.: 19/371,822

(22) Filed: Oct. 28, 2025

Related U.S. Application Data
(63) Continuation of application No. 30/013,664, filed on Jul. 17, 2025.
Application No. 30/013,664 is a continuation of application No. 30/001,615, filed on Apr. 29, 2025.

(51) Int. Cl. A61F 13/00; A61K 9/107; A61K 33/38; A61K 36/185; A61K 36/28; A61K 36/324; A61K 36/53; A61K 36/534; A61K 36/61; A61K 36/752; A61K 47/36; A61L 15/18; A61L 15/20; A61L 15/40; A61L 15/44

(52) U.S. Cl. CPC A61F 13/00059; A61F 13/00063; A61K 9/107; A61K 33/38; A61K 36/185; A61K 36/28; A61K 36/324; A61K 36/53; A61K 36/534; A61K 36/61; A61K 36/752; A61K 47/36; A61L 15/18; A61L 15/20; A61L 15/40; A61L 15/44; A61L 2300/104; A61L 2300/30; A61L 2300/404

(57) ABSTRACT

This disclosure provides wound healing compositions comprising glycerine, at least one or more emulsifiers selected from the group consisting of cetearyl glucoside, cetearyl alcohol, and phosphatidyl choline, at least one thickening agent selected from the group consisting of xanthan gum and gellan gum, at least one essential oil selected from the group consisting of tea tree oil, lavender oil, helichrysum oil, frankincense oil, menthol oil, peppermint oil, lavender oil, eucalyptus oil, chamomile oil, rose oil, geranium oil, and neroli oil, and chelated silver oxide, wherein said chelating agent is citric acid. The wound healing composition may be infused into a carrier material to form a wound dressing. The carrier material may be shaped to resemble a petal or a leaf of a plant known for wound-healing properties, such as plantain, comfrey, Calendula, Arnica, yarrow, or lavender. A method of treating a wound by applying the wound dressing is disclosed.

### CROSS-REFERENCE TO RELATED APPLICATIONS

This application claims priority to and is a utility application of U.S. Design Application Ser. No. 30/013,664, titled “Heart-Shaped Adhesive Covering”, filed Jul. 17, 2025, which is a continuation of U.S. Design Application No. Ser. No. 30/001,615, titled “Herbal Leaf Identifier Adhesive Covering(s)”, filed Apr. 29, 2025, which are hereby incorporated by reference in their entirety.

### FIELD OF INVENTION

The present disclosure relates to adhesive wound dressings, and more particularly to leaf and petal herbal-shaped adhesive wound dressings with impregnated or infused antimicrobial properties incorporating natural ingredients and plant-inspired designs.

### BACKGROUND

Wound healing is a complex biological process involving multiple stages, including inflammation, proliferation, and remodeling. Effective wound care plays a role in preventing infection, minimizing scarring, and promoting optimal healing outcomes. Traditional wound care products often rely on synthetic materials and chemical antimicrobial agents that can have limitations, such as potential toxicity, bacterial resistance development, and adverse reactions in sensitive individuals.

There is growing interest in developing wound care solutions that incorporate natural ingredients and biomimetic designs inspired by plants known for their healing properties. Certain herbs and botanicals have been used in traditional medicine for centuries to treat wounds and skin conditions. These natural remedies often contain compounds with antimicrobial, anti-inflammatory, and wound healing properties that may offer advantages over synthetic alternatives.

Adhesive wound dressings serve an important role in protecting wounds from external contaminants while maintaining a moist healing environment. However, conventional adhesive bandages typically have a standard rectangular shape that may not conform well to all wound types and body contours. There is room for innovation in both the shape of the adhesive dressing and the infusion on the pad within wound dressing designs that could enhance patient comfort and compliance while providing effective wound protection.

Additionally, increasing antibiotic resistance has led to renewed focus on developing alternative antimicrobial strategies for wound care. Natural antimicrobial compounds derived from plants offer potential advantages in terms of reduced likelihood of resistance development and may provide localized antimicrobial protection without systemic effects. The incorporation of such compounds into wound dressings could provide targeted therapeutic benefits at the wound site.

As environmental and health concerns drive interest in more natural and sustainable healthcare products, there are opportunities to reimagine wound care solutions. Combining insights from ethnobotany, materials science, and bioengineering could lead to novel wound dressing designs that harness the healing properties of plants while meeting modern medical standards for safety and efficacy. The development of wound dressings that integrate natural herbal extracts with innovative design features represents an area of ongoing research and development in the field of wound care management.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a wound healing composition is provided. The wound healing composition comprises an herbal glycerite consisting of at least one herb such as narrow leaf plantain (Plantago lanceolata), yarrow (Achillea millefolium), broadleaf plantain (Plantago major), comfrey (Symphytum officinale), chamomile (Chamomilla recutita), Calendula (Calendula officinalis), Arnica (Arnica montana), marsh mallow (Althaea officinalis L. (Malvaceae), St. John's Wort (Hypericum perforatum), lavender (Lavandula angustifolia), and comfrey (Symphytum officinale), aloe vera (Aloe barbadensis), at least one emulsifier selected from the group consisting of cetearyl glucoside and phosphatidyl choline, at least one thickening agent selected from the group consisting of xanthan gum and gellan gum, at least one essential oil selected from the group consisting of tea tree oil, lavender oil, helichrysum oil, and frankincense oil, menthol oil, peppermint oil, lavender oil, eucalyptus oil, chamomile oil, rose oil, geranium oil, and neroli oil, and chelated silver oxide, wherein the chelating agent is citric acid.

According to other aspects of the present disclosure, the wound healing composition may include one or more of the following features. The preferably plant based herbal Kosher USP glycerine may be present in an amount of 60-70% by weight of the composition. The at least one emulsifier may be present in an amount of 5-15% by weight of the composition. The at least one thickening agent may be present in an amount of 0.5-2% by weight of the composition. The at least one essential oil may be present in an amount of 1-5% by weight of the composition. The chelated silver oxide may be present in an amount of 0.1-5% by weight of the composition. The at least one essential oil may comprise a combination of essential oils listed above. tea tree oil and lavender oil.

According to another aspect of the present disclosure, a wound dressing is provided. The wound dressing comprises a carrier material and a wound healing composition infused into the carrier material, the wound healing composition comprising herbal infused glycerine, at least one emulsifier, at least one thickening agent, at least one essential oil, and an antimicrobial agent.

According to other aspects of the present disclosure, the wound dressing may include one or more of the following features. The carrier material may be selected from the group consisting of gauze, non-woven fabric, and hydrocolloid dressing. The at least one emulsifier may be selected from the group consisting of cetearyl glucoside and phosphatidyl choline. The at least one thickening agent may be selected from the group consisting of xanthan gum and gellan gum. The at least one essential oil may be selected from the group described above. consisting of tea tree oil, lavender oil, eucalyptus oil, helichrysum oil, chamomile oil, rose oil, geranium oil, neroli oil, and peppermint oil. The antimicrobial agent may comprise chelated silver oxide. The chelated silver oxide may be present in an amount of 0.1-5% by weight of the wound healing composition.

According to another aspect of the present disclosure, a method of treating a wound is provided. The method comprises applying to a wound a wound dressing comprising a carrier material infused with a wound healing composition, wherein the wound healing composition comprises glycerine, at least one emulsifier, at least one thickening agent, at least one essential oil, and an antimicrobial agent, and wherein the carrier material is shaped to resemble a leaf or petal of a plant known for its wound-healing properties. The leaf shape/bandage type is selected to provide the user with an easy reference regarding the type of wound healing plant based ointment composition exists with the user's selection.

According to other aspects of the present disclosure, the method may include one or more of the following features. The plant known for its wound-healing properties may be selected from the group consisting of plantain (both Plantago lanceolata and Plantago major), comfrey (Symphytum officinale), Calendula (Calendula officinalis), Arnica (Arnica montana), lavender (Lavendula angustifolia) and comfrey (Symphytum officinale). The carrier material shaped to resemble a narrow plantain leaf may comprise an elongated form with tapered ends and a central vein structure. The carrier material shaped to resemble a comfrey leaf and broadleaf plantain may comprise a broader, more oval shape with a mosaic pattern across its surface. The carrier material shaped to resemble a Calendula leaf may comprise serrated edges and a rounded overall form. The carrier material shaped to resemble a heart constructed from two petals of lavender may comprise a smaller, more heart-like shape with a mosaic pattern across its surface. The method may further comprise a display window incorporated into a central region of the Calendula leaf-shaped carrier material, wherein the display window allows for visual inspection of the covered wound area without removing the wound dressing.

Even more specifically the present disclosure provides for a wound healing composition, comprising:
glycerine;
at least one emulsifier selected from any one or more of a group consisting of cetearyl glucoside, cetearyl alcohol, and phosphatidyl choline;
at least one or more thickening agent selected from a group consisting of xanthan gum and gellan gum;
at least one or more essential oils selected from a group consisting of tea tree oil, lavender oil, helichrysum oil, frankincense oil, menthol oil, peppermint oil, lavender oil, eucalyptus oil, chamomile oil, rose oil, geranium oil, and neroli oil, tea tree oil, lavender oil, chamomile oil, and peppermint oil; and
chelated silver oxide, wherein the chelating agent is citric acid.
wherein the glycerine is present in an amount of 60-70% by weight of the composition.
wherein the at least one emulsifier is present in an amount of 5-15% by weight of the composition.
wherein the at least one emulsifier is cetearyl glucoside.
wherein the at least one thickening agent is present in an amount of 0.5-2% by weight of the composition.
wherein the at least one essential oil is present in an amount of 1-5% by weight of the composition.
wherein the at least one essential oil comprises a combination of the oils listed above is tea tree oil and lavender oil.
wherein the chelated silver oxide is present in an amount of 0.1-5% by weight of the composition.

Another embodiment includes a wound dressing, comprising:
a carrier material shaped to resemble a petal or a leaf of a plant known for wound-healing properties; and
a wound healing composition infused into the carrier material, the wound healing composition comprising glycerine, at least one emulsifier, at least one thickening agent, at least one essential oil, and an antimicrobial agent.
wherein the carrier material is selected from the group consisting of gauze, non-woven fabric, and hydrocolloid dressing.
wherein the plant known for wound-healing properties is selected from the group consisting of narrow leaf plantain (Plantago lanceolata), yarrow (Achillea millefolium), broadleaf plantain (Plantago major), comfrey (Symphytum officinale), chamomile (Chamomilla recutita), Calendula (Calendula officinalis), Arnica (Arnica montana), marsh mallow (Althaea officinalis L. (Malvaceae), St. John's Wort (Hypericum perforatum), lavender (Lavendula angustifolia). comfrey (Symphytum officinale), and aloe vera (Aloe barbadensis) plantain (Plantago lanceolata), comfrey (Symphytum officinale), Calendula (Calendula officinalis), and yarrow (Achillea millefolium).
wherein the carrier material is shaped to resemble a plantain leaf comprising an elongated form with tapered ends and a central vein structure.
wherein the carrier material is shaped to resemble a comfrey leaf.
wherein the carrier material is shaped to resemble Calendula leaf.
wherein the carrier material is shaped to resemble a comfrey leaf comprising a broader, oval shape with a mosaic pattern across its surface.
wherein the antimicrobial agent comprises chelated silver oxide with citric acid as a chelating agent and wherein the dressing is also a covering.

The present disclosure also provide for a method of treating skin irritations, comprising:
applying to a wound, abrasion, cut or scrape a dressing comprising a carrier material infused with a wound healing composition, wherein the dressing composition comprises glycerine, at least one emulsifier, at least one thickening agent, at least one essential oil, and an antimicrobial agent, and wherein the carrier material is shaped to resemble a petal or a leaf of a plant known for wound-healing properties.
wherein the plant known for wound-healing properties is selected from the group consisting of plantain (Plantago major), comfrey (Symphytum officinale), Calendula (Calendula officinalis), yarrow (Achillea millefolium), and lavender (Lavendula angustifolia).
wherein the carrier material is shaped to resemble a plantain leaf comprising an elongated form with tapered ends and a central vein structure.
wherein the carrier material is shaped to resemble a Calendula leaf comprising serrated edges and a rounded overall form with a display window incorporated into a central region, wherein the display window allows for visual inspection of the covered wound area without removing the wound dressing.
wherein the wound healing composition comprises tea tree oil and lavender oil as the at least one essential oil.
wherein the antimicrobial agent comprises chelated silver oxide with citric acid as a chelating agent, and wherein the chelated silver oxide is present in an amount of 0.1-5% by weight of the wound healing composition.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 is a top perspective view of the plantain (Plantago lanceolata) leaf shaped herbal identifier adhesive covering;

FIG. 2 is a bottom perspective view thereof;

FIG. 3 is a front view thereof;

FIG. 4 is a rear view thereof;

FIG. 5 is a left view of the plantain (Plantago lanceolata) leaf shaped herbal identifier adhesive covering of FIG. 4;

FIG. 6 is a right view thereof.

FIG. 7 is a top view thereof;

FIG. 8 is a bottom view thereof;

FIG. 9 is a top perspective view of the comfrey (Symphytum officinale) leaf shaped herbal identifier adhesive covering;

FIG. 10 is a bottom perspective view thereof;

FIG. 11 is a front view thereof;

FIG. 12 is a rear view thereof;

FIG. 13 is a left view of the comfrey (Symphytum officinale) leaf shaped herbal identifier adhesive covering of FIG. 12;

FIG. 14 is a right view thereof.

FIG. 15 is a top view thereof;

FIG. 16 is a bottom view thereof;

FIG. 17 is a top perspective view of a Calendula (Calendula officinalis) leaf shaped herbal identifier adhesive covering;

FIG. 18 is a bottom perspective view thereof;

FIG. 19 is a front view thereof;

FIG. 20 is a rear view thereof;

FIG. 21 is a left view of a Calendula (Calendula officinalis) leaf shaped herbal identifier adhesive covering of FIG. 20;

FIG. 22 is a right view thereof;

FIG. 23 is a top view thereof;

FIG. 24 is a bottom view thereof;

FIG. 25 is a top perspective view of an Arnica (Arnica montana) leaf shaped herbal identifier adhesive covering;

FIG. 26 is a bottom perspective view thereof;

FIG. 27 is a front view thereof;

FIG. 28 is a rear view thereof;

FIG. 29 is a left view of an Arnica (Arnica montana) leaf shaped herbal identifier adhesive covering of FIG. 28;

FIG. 30 is a right view thereof.

FIG. 31 is a top view thereof;

FIG. 32 is a bottom view thereof;

FIG. 33 is a top perspective view of the heart-shaped adhesive covering formed from two flower petals of Lavender (Lavendula angustifolia;

FIG. 34 is a bottom perspective view of the heart-shaped adhesive covering;

FIG. 35 is front view thereof;

FIG. 36 is a rear view of the heart-shaped adhesive covering of FIG. 1;

FIG. 37 is the left view of an embodiment of the heart-shaped adhesive covering as shown in FIG. 4;

FIG. 38 is a right view thereof;

FIG. 39 is a top view thereof;

FIG. 40 is a bottom view thereof;

FIG. 41 is a top perspective view of the broadleaf plantain (Plantago major) leaf shaped herbal identifier adhesive covering;

FIG. 42 is a bottom perspective view thereof;

FIG. 43 is a front view thereof;

FIG. 44 is a rear view thereof;

FIG. 45 is a left view of the broadleaf plantain (Plantago major) leaf shaped herbal identifier adhesive covering of FIG. 4;

FIG. 46 is a right view thereof.

FIG. 47 is a top view thereof;

FIG. 48 is a bottom view thereof;

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

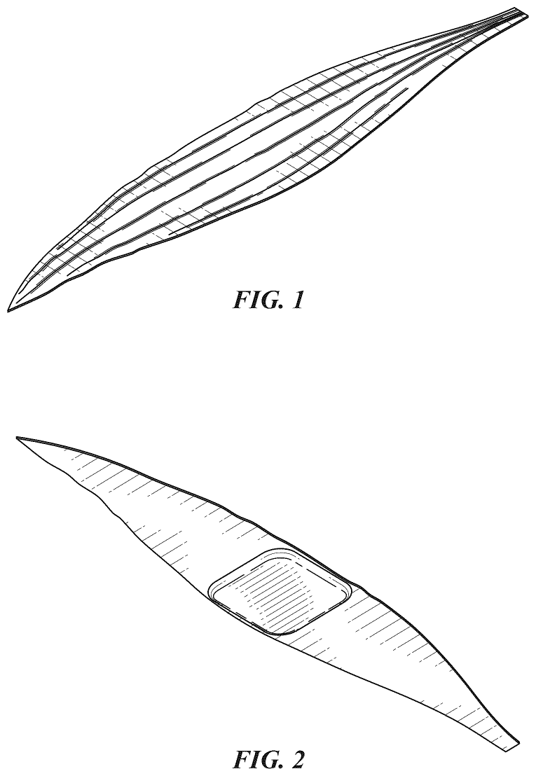

The present disclosure relates to a wound dressing system that incorporates natural herbal properties with leaf-inspired designs for wound care applications (FIGS. 1, 9, 17, 25, 33). The wound dressing may comprise a carrier material shaped to resemble a leaf of a plant known for wound-healing properties and a wound healing composition infused into the carrier material. The carrier material may be configured to mimic the natural contours and structural features of medicinal plant leaves, creating a biomimetic approach to wound coverage. In some cases, the wound healing composition may include glycerine as a base component along with various active ingredients that support the wound healing process.

The herbal-shaped designs of the carrier material may serve multiple functional purposes beyond aesthetic appeal. In some cases, the herbal-shaped designs may serve as visual identifiers to aid in selection and application of appropriate coverings for different wound types or healing stages. The distinctive leaf shapes may allow healthcare providers and patients to quickly identify specific wound dressing formulations based on the plant species represented by the carrier material design. For example, a carrier material shaped to resemble a plantain leaf may indicate a formulation with specific antimicrobial properties, while a comfrey leaf-shaped carrier material may signify enhanced tissue regeneration capabilities.

The wound healing composition infused into the carrier material may contain a combination of natural and synthetic components designed to create an optimal healing environment. The composition may include emulsifiers that help stabilize the formulation and ensure uniform distribution of active ingredients throughout the carrier material. Thickening agents may be incorporated to adjust the viscosity and consistency of the composition, allowing for proper absorption and retention within the carrier material structure. Essential oils derived from medicinal plants may provide antimicrobial, anti-inflammatory, and analgesic properties that complement the wound healing process.

The integration of the wound healing composition with the herbal-shaped carrier material may create a synergistic effect that enhances both the therapeutic and practical aspects of wound care. The leaf-inspired shapes may allow for better conformity to various body contours and wound geometries, potentially improving coverage and adherence compared to conventional rectangular bandages. The natural edges and contours of the leaf shapes may distribute pressure more evenly across the wound site, reducing the risk of irritation or damage to surrounding healthy tissue. The combination of shape and composition may work together to provide a comprehensive wound care solution that addresses multiple aspects of the healing process simultaneously.

The wound healing composition may serve as the therapeutic foundation of the wound dressing system, incorporating multiple components that work synergistically to support the wound healing process. The composition may include glycerine as a primary base component, which may function as a humectant to maintain moisture at the wound site and create an environment conducive to cellular regeneration. In some cases, the glycerine may be present in an amount of 60-70% by weight of the composition, providing a substantial foundation that helps deliver other active ingredients while maintaining the desired consistency and therapeutic properties. The glycerine component may also contribute to the biocompatibility of the formulation, as glycerine may be well-tolerated by various skin types and wound conditions.

The wound healing composition may incorporate at least one emulsifier selected from the group consisting of cetearyl glucoside and phosphatidyl choline to enhance the stability and uniformity of the formulation. In some cases, at least one emulsifier may be present in an amount of 5-15% by weight of the composition, providing adequate stabilization without compromising the therapeutic properties of other components. Cetearyl glucoside may function as a non-ionic surfactant derived from natural sources, helping to create stable emulsions between the glycerine base and oil-soluble ingredients such as essential oils. The cetearyl glucoside may facilitate the uniform distribution of active ingredients throughout the composition while maintaining compatibility with sensitive wound environments. Alternatively, phosphatidyl choline may serve as an emulsifier in certain formulations, where phosphatidyl choline may aid in the dispersion of lipophilic compounds and enhance the penetration of active ingredients into wound tissue.

Thickening agents may be incorporated into the wound healing composition to adjust viscosity and create the appropriate consistency for infusion into carrier materials. This includes infusion of any and all portions of the composition into a gauze portion of the bandage that often is made from cotton or other manmade fabrics such as polyester staple. The composition may include at least one thickening agent selected from the group consisting of xanthan gum and gellan gum, where these agents may help achieve the desired gel-like texture that facilitates proper absorption and retention within the wound dressing structure. In some cases, the at least one thickening agent may be present in an amount of 0.5-2% by weight of the composition, providing adequate thickening properties while allowing for easy application and removal of the wound dressing. Xanthan gum may function as a polysaccharide-based thickening agent that forms stable gels when combined with the glycerine base and other aqueous components. The xanthan gum may contribute to the overall stability of the formulation and help maintain consistent viscosity across different storage conditions. Gellan gum may serve as an alternative thickening agent that forms thermoreversible gels, allowing for temperature-dependent viscosity changes that may facilitate manufacturing processes and application procedures.

The wound healing composition may include at least one essential oil selected from the group consisting of tea tree oil, lavender oil, eucalyptus oil, chamomile oil, and peppermint oil to provide therapeutic benefits that complement the wound healing process. In some cases, the at least one essential oil may be present in an amount of 1-5% by weight of the composition, providing therapeutic concentrations while maintaining safety and tolerability for topical application. Tea tree oil may exhibit antimicrobial properties that help prevent bacterial colonization at the wound site, while lavender oil may provide anti-inflammatory and analgesic effects that reduce pain and swelling associated with wound healing. The combination of tea tree oil and lavender oil may create synergistic effects that enhance both antimicrobial protection and patient comfort during the healing process. Helichrysum oil may contribute additional antimicrobial and anti-inflammatory properties, while chamomile oil may provide soothing effects that help calm irritated skin around the wound area. Peppermint oil may offer cooling sensations and antimicrobial benefits that complement the therapeutic profile of the composition.

Antimicrobial protection may be enhanced through the incorporation of chelated silver oxide, where the chelating agent may be citric acid to improve stability and bioavailability of the silver component. In some cases, the chelated silver oxide may be present in an amount of 0.1-5% by weight of the composition, providing broad-spectrum antimicrobial activity against various pathogens that could potentially compromise wound healing. The chelation process with citric acid may enhance the solubility and stability of the silver oxide, allowing for more controlled release of antimicrobial agents at the wound site. The chelated silver oxide may provide sustained antimicrobial protection throughout the wear time of the wound dressing, helping to maintain a clean wound environment that supports natural healing processes. The citric acid chelating agent may also contribute mild acidic properties that help maintain an optimal pH environment for wound healing while enhancing the overall stability of the silver oxide component within the glycerine-based matrix.

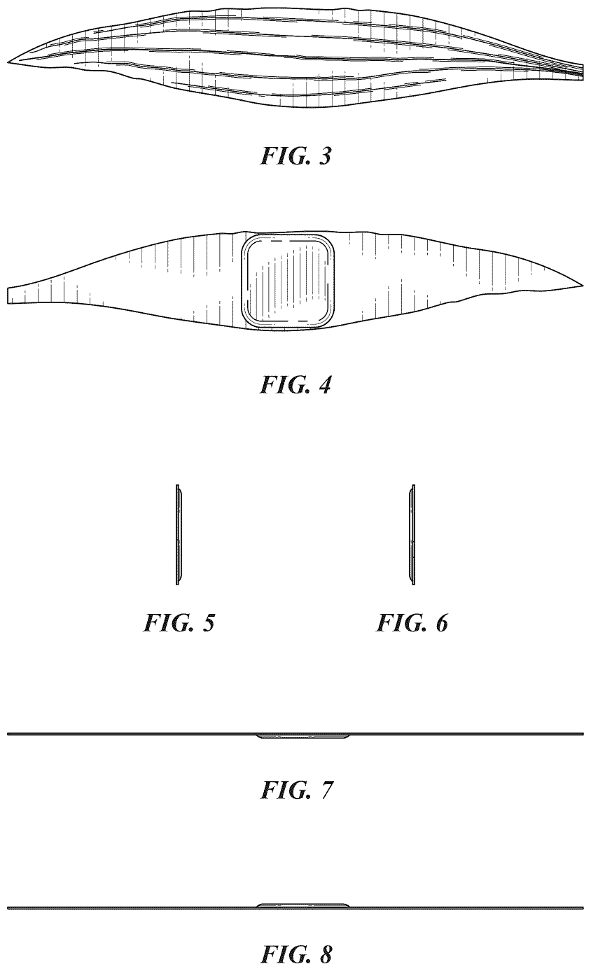

The carrier material may be shaped, as in FIG. 1 shown from a top perspective view, to resemble a plantain leaf, an example of which is Plantago lanceolata, where the plantain leaf design may incorporate specific structural features that enhance both the aesthetic and functional aspects of the wound dressing. In some cases, the carrier material shaped to resemble a plantain leaf may comprise an elongated form with tapered ends and a central vein structure that mimics the natural morphology of Plantago lanceolata leaves. The elongated form may provide enhanced coverage for linear wounds or wounds located on body areas that benefit from directional protection, such as along limbs or across joint areas. The tapered ends of the plantain leaf-shaped carrier material may facilitate smooth application and removal while reducing the likelihood of edge lifting or premature detachment during wear. The central vein structure may serve both aesthetic and functional purposes, creating a visual representation of the natural plantain leaf while providing structural reinforcement to the carrier material. Additional views of the Plantain lanceolata shaped adhesive wound covering are provided showing a bottom perspective view (FIG. 2), front view (FIG. 3), rear view (FIG. 4), left view (FIG. 5), right view (FIG. 6), top view (FIG. 7), and bottom view (FIG. 8).

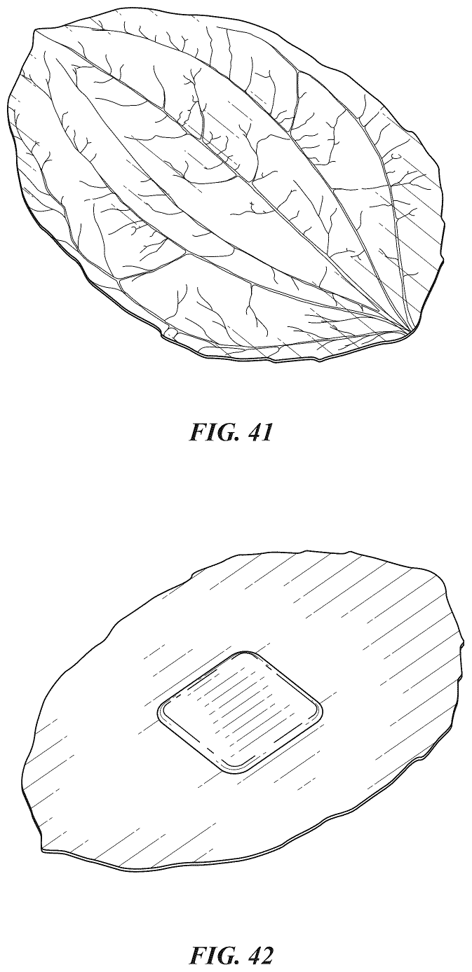

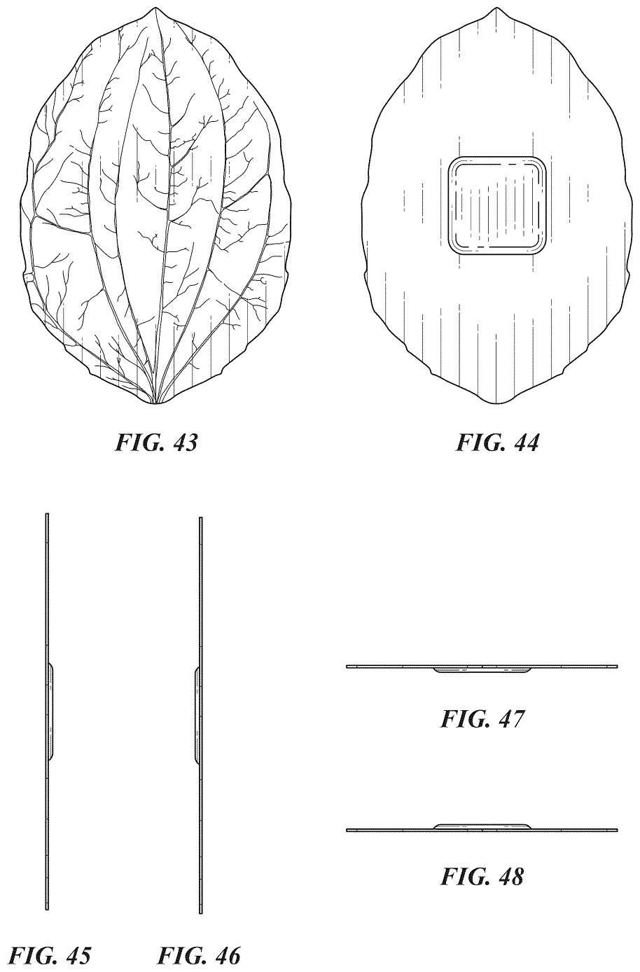

In an additional embodiment, the leaf and herbal component can be chosen from another member of plantain family, particularly Plantago major. The carrier material may be shaped, as in FIG. 41 shown from a top perspective view, to resemble a plantain leaf, an example of which is Plantago lanceolata, where the plantain leaf design may incorporate specific structural features that enhance both the aesthetic and functional aspects of the wound dressing. Additional views of the Plantain lanceolata shaped adhesive wound covering are provided showing a bottom perspective view (FIG. 42), front view (FIG. 43), rear view (FIG. 44), left view (FIG. 45), right view (FIG. 46), top view (FIG. 47), and bottom view (FIG. 48).

The plantain species, particularly Plantago lanceolata, may be recognized for wound-healing properties that have been utilized in traditional medicine applications for centuries. Plantain leaves may contain biologically active compounds such as polysaccharides, lipids, caffeic acid derivatives, flavonoids, iridoid glycosides, and terpenoids that contribute to wound healing activity. The polysaccharides present in plantain may provide moisturizing and protective properties that help maintain optimal wound hydration levels. Caffeic acid derivatives may offer antioxidant benefits that protect wound tissue from oxidative stress during the healing process. Flavonoids found in plantain may contribute anti-inflammatory properties that help reduce swelling and irritation at the wound site. The iridoid glycosides may provide antimicrobial effects that help prevent bacterial colonization and infection in wound environments.

The wound healing composition infused into the plantain leaf-shaped carrier material may include herbal glycerites made from specific medicinal plants including plantain, comfrey, Calendula, lavender, and yarrow as additional active ingredients that complement the therapeutic profile of the wound dressing. The plantain glycerite may be prepared using a specific process involving a 75% glycerin and 25% distilled water ratio, with a 3:1 liquid to herb ratio for dried plantain material. The plantain herb material may be heated gently in a double boiler for at least 40 minutes to extract the bioactive compounds into the glycerin-water mixture. The resulting plantain glycerite may contain concentrated levels of the wound-healing compounds naturally present in plantain leaves, including the polysaccharides, flavonoids, and other therapeutic constituents that support tissue regeneration and wound closure.

The central vein structure of the plantain leaf-shaped carrier material may incorporate functional elements where the central vein or midrib corresponds to a region of increased absorbency or medication delivery. In some cases, the central vein area may be designed with enhanced porosity or modified material density that allows for greater absorption of wound exudate or more concentrated delivery of therapeutic agents from the wound healing composition. The central vein region may serve as a primary channel for distributing the wound healing composition throughout the carrier material, ensuring that active ingredients reach all areas of the wound site. The vein structure may also provide mechanical reinforcement to the carrier material, helping to maintain structural integrity during application and wear while allowing for flexibility and conformability to wound contours.

The combination of the plantain leaf shape with the herbal glycerite composition may create a biomimetic wound dressing that leverages both the physical properties of the leaf design and the therapeutic properties of plantain-derived compounds. The elongated form with tapered ends may allow the wound dressing to conform to various wound geometries and body contours, providing comprehensive coverage while maintaining secure adherence. The central vein structure may facilitate even distribution of the wound healing composition across the wound site, ensuring that therapeutic agents reach all areas of the wound bed. The plantain glycerite component may provide sustained release of bioactive compounds that support multiple aspects of wound healing, including antimicrobial protection, anti-inflammatory effects, and tissue regeneration promotion. The synergistic combination of shape and composition may result in a wound dressing that addresses both the physical and biochemical requirements for optimal wound healing outcomes.

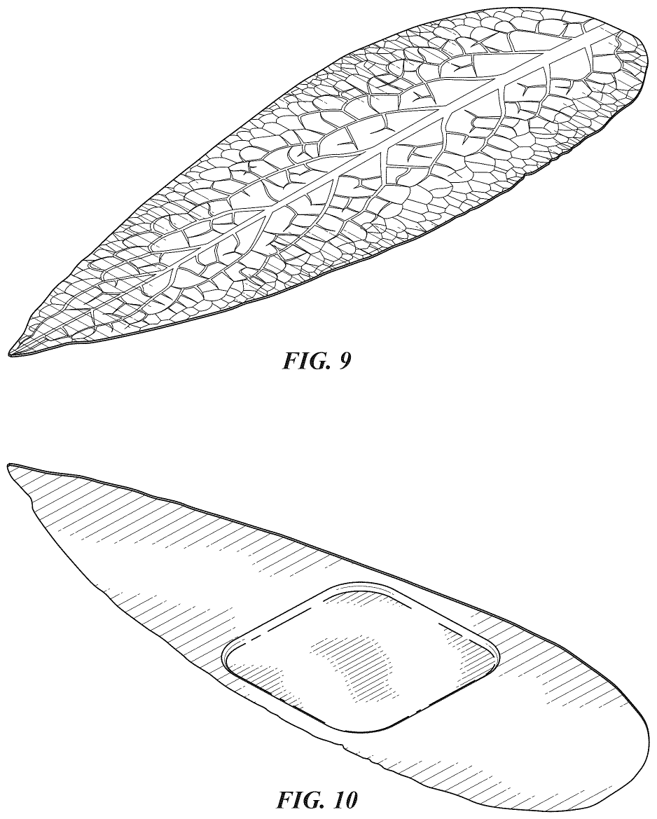

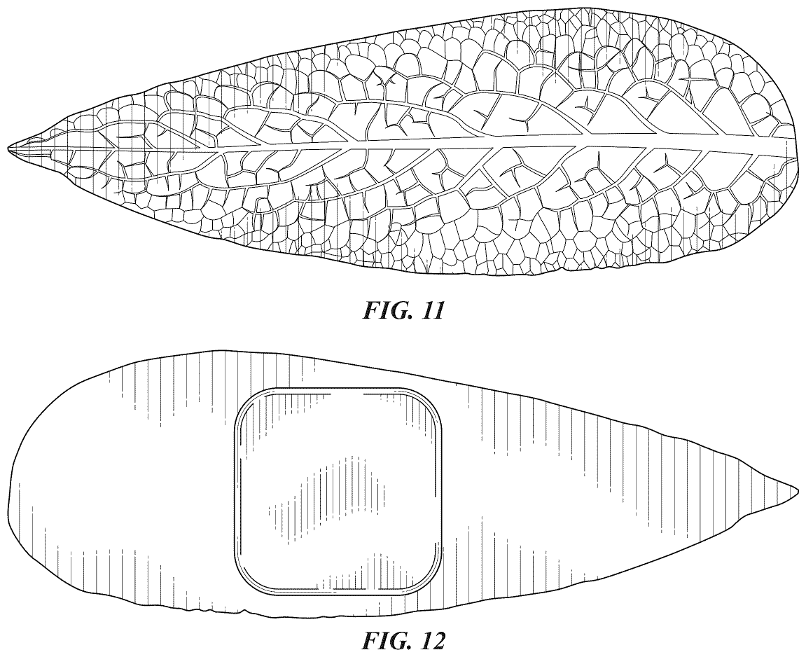

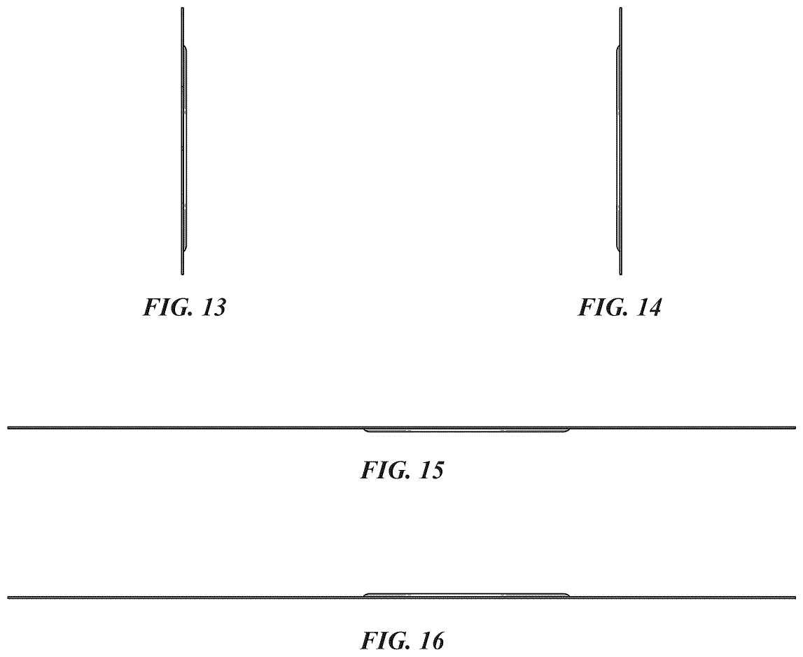

The carrier material may be shaped to resemble a comfrey (Symphytum officinale) leaf (FIG. 9, top perspective view), where the comfrey leaf design may incorporate a broader, oval shape that distinguishes the comfrey-inspired wound dressing from other herbal leaf configurations. In some cases, the carrier material shaped to resemble a comfrey leaf may comprise a broader, oval shape with a mosaic pattern across the surface that mimics the natural cellular structure and texture characteristics of Symphytum officinale leaves. The broader oval configuration may provide enhanced coverage area compared to more elongated leaf shapes, making the comfrey leaf-shaped carrier material particularly suitable for wounds that require comprehensive surface protection or wounds located on body areas with curved contours. The oval geometry may facilitate conformability to rounded wound sites while maintaining structural integrity during application and wear periods. The broader dimensions of the comfrey leaf shape may allow for greater infusion capacity of the wound healing composition, potentially providing enhanced therapeutic agent delivery across larger wound areas. Additional views of the Symphytum officinale shaped adhesive wound covering are provided showing a bottom perspective view (FIG. 10), front view (FIG. 11), rear view (FIG. 12), left view (FIG. 13), right view (FIG. 14), top view (FIG. 15), and bottom view (FIG. 16).

The mosaic pattern incorporated across the surface of the comfrey leaf-shaped carrier material may comprise irregular cell-like segments that mimic the natural texture of a comfrey leaf and provide both aesthetic and functional benefits to the wound dressing design. In some cases, the mosaic pattern may consist of interconnected polygonal segments of varying sizes that replicate the cellular architecture observed in natural comfrey leaf tissue. The irregular cell-like segments may create a textured surface that enhances the absorption and retention characteristics of the wound healing composition within the carrier material structure. The mosaic pattern may also provide mechanical advantages by creating multiple zones of flexibility that allow the carrier material to conform to complex wound geometries and body contours without compromising the integrity of the overall dressing structure. The cell-like segments may function as individual reservoirs for the wound healing composition, allowing for controlled release of therapeutic agents as the segments interact with wound exudate and moisture at the application site.

The comfrey species, particularly Symphytum officinale, may be recognized for wound-healing properties that have been documented in traditional medicine applications and modern phytochemical research. Comfrey may contain allantoin as a primary bioactive compound, where allantoin may function as a protein that promotes cell renewal, tissue regeneration, and the formation of new bone tissue in wound healing applications. The allantoin content in comfrey may provide regenerative effects that accelerate the proliferation phase of wound healing by stimulating cellular division and promoting the development of new tissue structures. Mucilage may represent another major constituent of comfrey, where the mucilage may be composed of fructose and glucose units that contribute moisturizing and protective properties to wound environments. The mucilage component may help maintain optimal hydration levels at the wound site while providing a protective barrier against external contaminants and mechanical irritation.

The wound healing composition infused into the comfrey leaf-shaped carrier material may include comfrey glycerite as an additional active ingredient that concentrates the therapeutic compounds naturally present in comfrey plant material. The comfrey glycerite may be prepared using a specific process involving a 75% glycerin and 25% distilled water ratio, with a 3:1 liquid to herb ratio for dried comfrey material, where the comfrey herb material may be heated gently in a double boiler for at least 40 minutes to extract the bioactive compounds. The resulting comfrey glycerite may contain concentrated levels of allantoin, mucilage, and rosmarinic acid that support tissue regeneration and wound closure processes. Rosmarinic acid may function as a phenolic acid found in comfrey that provides antioxidant and anti-inflammatory properties, helping to protect wound tissue from oxidative damage while reducing inflammatory responses that could impede healing progress. The combination of allantoin, mucilage, and rosmarinic acid in the comfrey glycerite may create synergistic effects that enhance the overall therapeutic profile of the wound healing composition.

The broader oval shape of the comfrey leaf-shaped carrier material may provide enhanced surface area for therapeutic agent distribution while accommodating the mosaic pattern design that facilitates controlled release of the wound healing composition. The oval geometry may allow for optimal placement over wounds that require comprehensive coverage, such as abrasions, burns, or surgical sites that benefit from uniform therapeutic agent distribution across the entire wound bed. The mosaic pattern may work in conjunction with the oval shape to create multiple zones of therapeutic activity, where each cell-like segment may function as an individual delivery system for the comfrey-enriched wound healing composition. The irregular nature of the cell-like segments may provide varying release rates across different areas of the carrier material, potentially allowing for customized therapeutic delivery based on the specific requirements of different wound regions. The combination of the broader oval shape with the mosaic pattern may result in a wound dressing that maximizes both coverage area and therapeutic efficacy while maintaining the biomimetic characteristics that distinguish the comfrey leaf-inspired design from conventional wound dressing configurations.

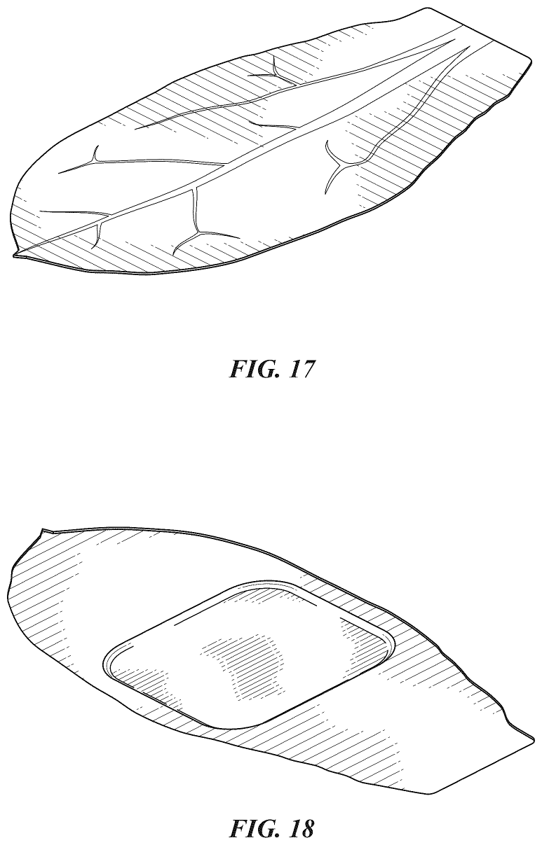

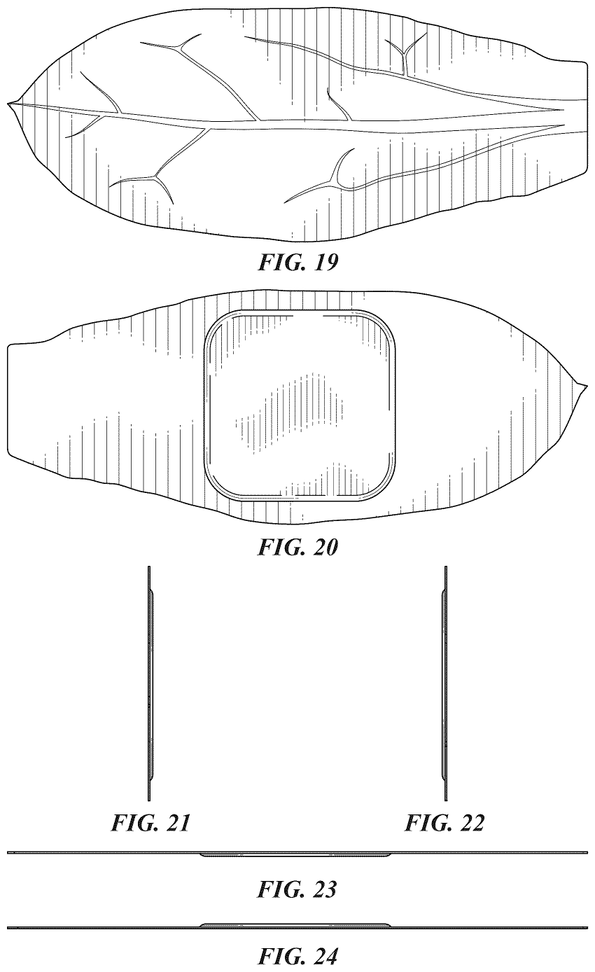

The carrier material may be shaped to resemble a Calendula (Calendula officinalis) leaf (FIG. 17, shown from a top perspective view), where the Calendula leaf design may incorporate serrated edges and a rounded overall form that distinguishes the Calendula-inspired wound dressing from other herbal leaf configurations. In some cases, the carrier material shaped to resemble a Calendula leaf may comprise serrated edges that replicate the natural dentate margin characteristics observed in Calendula officinalis leaves, where the serrated pattern may provide enhanced flexibility and conformability to wound contours. The rounded overall form of the Calendula leaf-shaped carrier material may facilitate application over curved body surfaces and circular or oval wound geometries, where the rounded configuration may distribute mechanical stress more evenly across the carrier material structure during movement and wear. The serrated edges may create multiple contact points that enhance adherence to skin surfaces while allowing for gradual pressure distribution that reduces the likelihood of edge lifting or premature detachment. The combination of serrated edges with the rounded form may result in a carrier material design that balances structural integrity with flexibility, allowing the Calendula leaf-shaped wound dressing to maintain secure placement while accommodating natural body movements and wound site dynamics. Additional views of the Calendula officinale shaped adhesive wound covering are provided showing a bottom perspective view (FIG. 18), front view (FIG. 19), rear view (FIG. 20), left view (FIG. 21), right view (FIG. 22), top view (FIG. 23), and bottom view (FIG. 24).

The Calendula leaf-shaped carrier material may incorporate a display window in the central region that allows visual inspection of the wound without removing the dressing, where the display window may function as a transparent or translucent aperture that provides direct visual access to the wound bed. In some cases, the display window may be positioned within the central region of the rounded Calendula leaf form, where the central placement may correspond to the primary wound area while maintaining the therapeutic benefits of the surrounding carrier material and wound healing composition. The display window may be constructed from biocompatible transparent materials that maintain optical clarity while providing barrier protection against external contaminants and moisture loss. The size and shape of the display window may be configured to accommodate various wound dimensions while preserving the structural integrity of the surrounding Calendula leaf-shaped carrier material. The display window feature may allow healthcare providers and patients to monitor wound healing progress, assess exudate levels, and evaluate tissue regeneration without disrupting the wound environment through premature dressing removal.

The display window incorporated into the central region of the Calendula leaf-shaped carrier material may allow for visual inspection of the covered wound area without removing the wound dressing, where the inspection capability may facilitate continuous wound monitoring throughout the wear period of the dressing. In some cases, the display window may enable assessment of wound healing indicators such as tissue color changes, granulation tissue formation, epithelialization progress, and exudate characteristics without compromising the sterile wound environment maintained by the surrounding carrier material. The visual inspection capability may reduce the frequency of dressing changes required for wound assessment purposes, potentially minimizing trauma to healing tissue and reducing healthcare costs associated with frequent dressing replacements. The display window may be designed with optical properties that provide clear visualization under various lighting conditions while maintaining the barrier functions against bacterial penetration and moisture loss. The transparent or translucent nature of the display window material may allow for photographic documentation of wound healing progress, enabling healthcare providers to maintain visual records of treatment outcomes and healing trajectories over time.

The Calendula species, particularly Calendula officinalis, may be recognized for wound-healing properties that have been documented in traditional medicine applications and contemporary phytochemical research studies. Calendula flowers may contain triterpenoids, polyphenols, and carotenoids as primary bioactive constituents that contribute to the medicinal characteristics associated with wound healing applications. The triterpenoids present in Calendula may provide anti-inflammatory effects that help reduce swelling and inflammatory responses at wound sites, where the anti-inflammatory activity may facilitate the transition from the inflammatory phase to the proliferative phase of wound healing. Polyphenols found in Calendula may contribute antioxidant properties that protect wound tissue from oxidative stress and free radical damage during the healing process, where the antioxidant effects may help preserve cellular integrity and support optimal conditions for tissue regeneration. Carotenoids in Calendula may provide additional antioxidant benefits while contributing to the characteristic orange and yellow pigmentation associated with Calendula flowers, where the carotenoid compounds may also support cellular repair mechanisms and tissue remodeling processes.

The wound healing composition infused into the Calendula leaf-shaped carrier material may include Calendula glycerite as an additional active ingredient that concentrates the therapeutic compounds naturally present in Calendula flower material. The Calendula herbal glycerite may be prepared using a specific process involving a 75% glycerin and 25% distilled water ratio, with a 3:1 liquid to herb ratio for dried herb Calendula flowers, where the herb Calendula material may be heated gently in a double boiler for at least 40 minutes to extract the bioactive compounds into the glycerin-water mixture. The resulting Calendula herbal glycerite may contain concentrated levels of triterpenoids, polyphenols, and carotenoids that provide anti-inflammatory, antimicrobial, and tissue regeneration properties to the wound healing composition. The herbal Calendula glycerite may exhibit antimicrobial properties that help fight bacteria and antifungal properties that protect against fungal colonization at wound sites, where the antimicrobial effects may complement the chelated silver oxide component of the wound healing composition to provide comprehensive pathogen protection. The anti-inflammatory properties derived from the flavonoids present in Calendula may help reduce tissue swelling and inflammatory responses that could impede wound healing progress, while the tissue regeneration properties may support the formation of new tissue structures and promote wound closure processes.

The combination of the Calendula leaf shape with the display window feature and Calendula-enriched wound healing composition may create a comprehensive wound monitoring and treatment system that addresses both therapeutic and diagnostic aspects of wound care. The serrated edges and rounded form of the Calendula leaf-shaped carrier material may provide optimal conformability and adherence characteristics while the display window enables continuous visual assessment of wound healing progress without compromising the therapeutic environment. The Calendula glycerite component may deliver sustained anti-inflammatory and antimicrobial benefits that support multiple phases of wound healing, from initial inflammatory control through tissue regeneration and remodeling. The display window may facilitate evidence-based wound care decisions by allowing healthcare providers to observe healing indicators and adjust treatment protocols based on visual assessment of wound status. The integration of shape, function, and composition in the Calendula leaf-shaped wound dressing may result in a wound care solution that combines therapeutic efficacy with practical monitoring capabilities, potentially improving both clinical outcomes and patient satisfaction through enhanced wound care management.

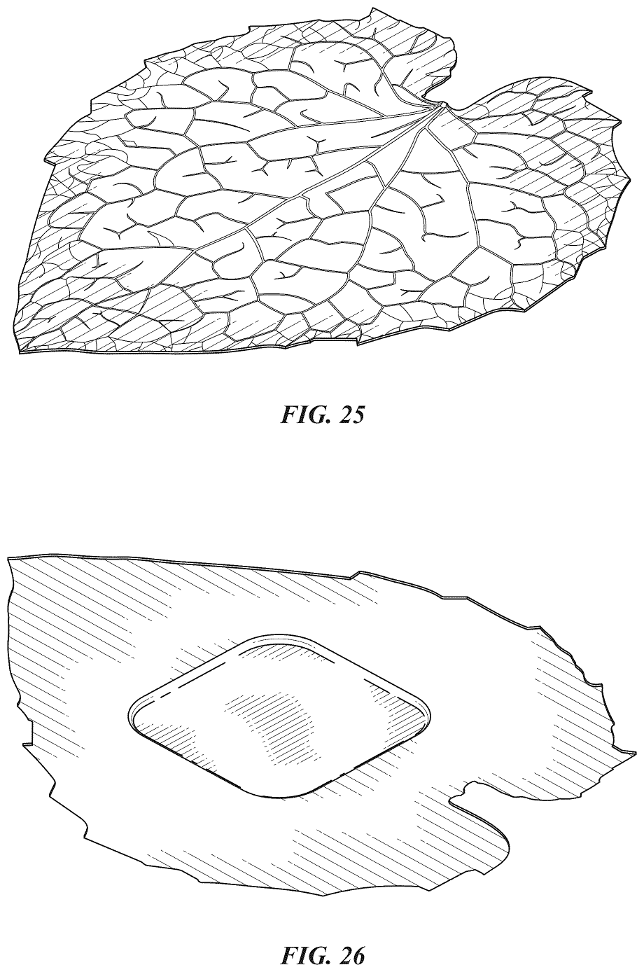

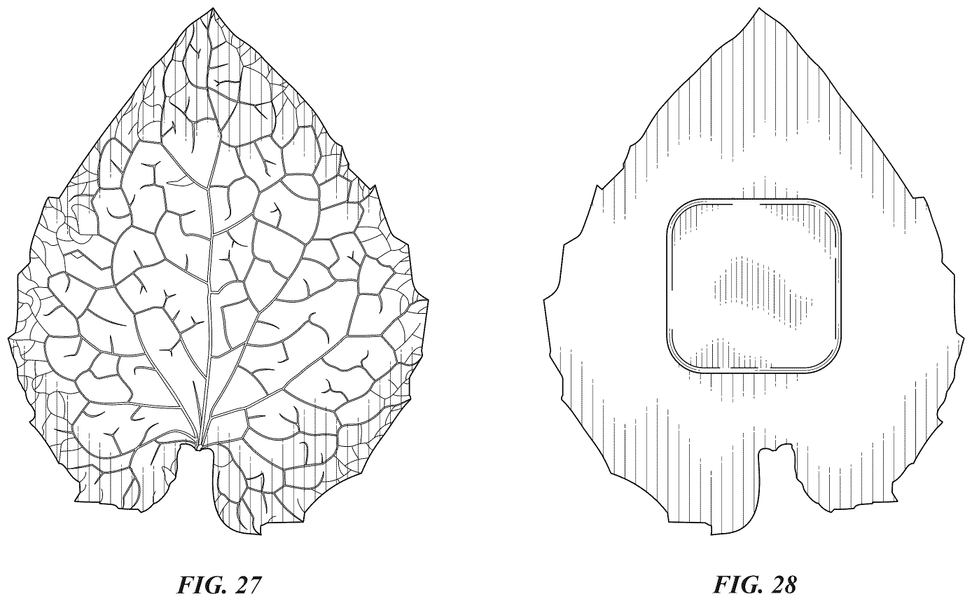

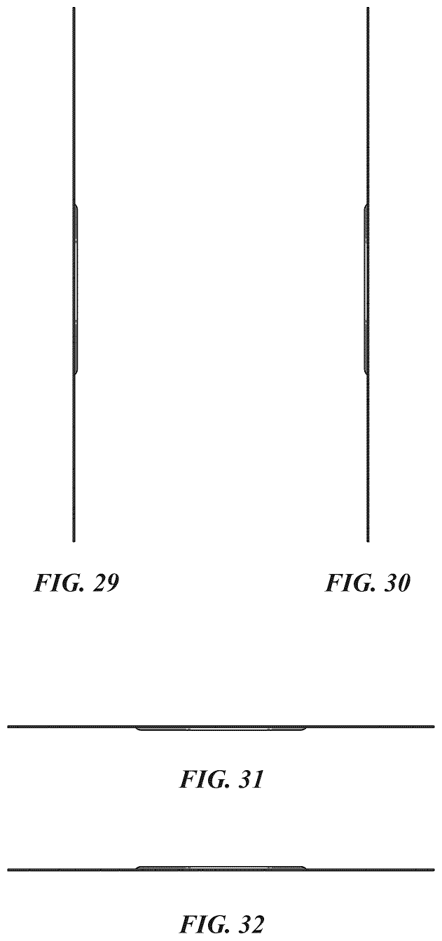

The carrier material may be shaped to resemble an Arnica (Arnica montana) leaf (FIG. 25) with an elongated oval form and slightly irregular edges including a leaf vein pattern extending from a central midrib that distinguishes the Arnica-inspired wound dressing from other herbal leaf configurations. In some cases, the carrier material shaped to resemble an Arnica leaf may comprise an elongated oval form that provides directional coverage characteristics suitable for wounds located along limbs or across areas that benefit from longitudinal protection. The elongated oval geometry may facilitate application over linear wounds, surgical incisions, or abrasions that extend across body surfaces in a directional manner. The slightly irregular edges of the Arnica leaf-shaped carrier material may replicate the natural margin variations observed in Arnica montana leaves, where the irregular edge pattern may provide enhanced flexibility and conformability to wound contours compared to geometric shapes with uniform edges. The irregular edges may create multiple contact points that distribute adherence forces across varying surface areas, potentially reducing localized pressure concentrations that could cause discomfort or skin irritation during wear periods. Additional views of the Arnica montana shaped adhesive wound covering are provided showing a bottom perspective view (FIG. 26), front view (FIG. 27), rear view (FIG. 28), left view (FIG. 29), right view (FIG. 30), top view (FIG. 31), and bottom view (FIG. 32).

The leaf vein pattern extending from a central midrib may provide both structural and functional benefits to the Arnica leaf-shaped carrier material design. In some cases, the leaf vein pattern may consist of branching channels that radiate outward from the central midrib toward the irregular edges of the elongated oval form, creating a network of pathways that facilitate distribution of the wound healing composition throughout the carrier material structure. The central midrib may function as a primary distribution channel for therapeutic agents, where the midrib region may be designed with enhanced porosity or modified material density that allows for concentrated delivery of active ingredients to the wound site. The branching vein pattern may create secondary distribution pathways that ensure uniform coverage of the wound healing composition across the entire surface area of the Arnica leaf-shaped carrier material. The vein structure may also provide mechanical reinforcement to the carrier material, helping to maintain structural integrity during application and wear while allowing for flexibility that accommodates natural body movements and wound site dynamics.

The Arnica species, particularly Arnica montana, may be recognized for wound-healing and pain-relieving properties that have been documented in traditional medicine applications and contemporary phytochemical research studies. Arnica may contain achilleine as a primary bioactive component, where achilleine may exhibit anti-inflammatory and astringent properties that make Arnica suitable for sensitive, irritated, or inflamed skin conditions. The anti-inflammatory effects of achilleine may help reduce swelling and inflammatory responses at wound sites, where the anti-inflammatory activity may facilitate the transition from the inflammatory phase to the proliferative phase of wound healing. The astringent properties of achilleine may contribute to tissue tightening effects that help control bleeding and reduce exudate production at wound sites. Arnica may function as a styptic agent that helps stop bleeding when applied directly to wounds, where the hemostatic effects may provide immediate benefits for acute wounds or wounds with active bleeding. The pain-relieving properties of Arnica may provide analgesic effects that reduce discomfort associated with wound healing processes, where the analgesic activity may improve patient comfort and compliance with wound care regimens.

The wound healing composition infused into the Arnica leaf-shaped carrier material may include Arnica as a pain reliever component in addition to other herbal pain-relieving plants that complement the therapeutic profile of the wound dressing. The Arnica component may be incorporated as an Arnica glycerite prepared using a specific process involving a 75% glycerin and 25% distilled water ratio, with a 3:1 liquid to herb ratio for dried Arnica material, where the Arnica herb material may be heated gently in a double boiler for at least 40 minutes to extract the bioactive compounds into the glycerin-water mixture. The resulting Arnica glycerite may contain concentrated levels of achilleine and other therapeutic compounds that provide anti-inflammatory, astringent, and analgesic properties to the wound healing composition. The Arnica glycerite may work synergistically with other pain-relieving herbal components to create a comprehensive analgesic effect that addresses multiple pathways of pain perception and inflammatory response. The combination of Arnica with other herbal pain-relieving plants may provide sustained pain relief throughout the wear period of the wound dressing, where the sustained analgesic effects may reduce the need for systemic pain medications and improve overall patient comfort during wound healing.

The elongated oval form of the Arnica leaf-shaped carrier material may provide enhanced coverage for wounds that require directional protection while accommodating the leaf vein pattern that facilitates controlled distribution of the Arnica-enriched wound healing composition. The oval geometry may allow for optimal placement over wounds that extend along body contours, such as surgical incisions, lacerations, or abrasions that follow anatomical lines or result from directional trauma. The slightly irregular edges may work in conjunction with the elongated form to create a carrier material design that balances structural coverage with natural conformability, allowing the Arnica leaf-shaped wound dressing to maintain secure placement while accommodating the dynamic movements associated with different body locations. The leaf vein pattern extending from the central midrib may create multiple zones of therapeutic activity, where each vein pathway may function as a distribution channel for the Arnica-enriched wound healing composition. The branching nature of the vein pattern may provide varying delivery rates across different areas of the carrier material, potentially allowing for customized therapeutic delivery based on the specific pain relief and anti-inflammatory requirements of different wound regions. The combination of the elongated oval shape with the irregular edges and vein pattern may result in a wound dressing that maximizes both directional coverage and analgesic efficacy while maintaining the biomimetic characteristics that distinguish the Arnica leaf-inspired design from conventional wound dressing configurations.

The carrier material may be selected from the group consisting of gauze, non-woven fabric, and hydrocolloid dressing, where each carrier material type may provide distinct structural and functional characteristics that influence the performance of the wound dressing system. Gauze may function as a traditional carrier material that provides a woven or non-woven fibrous structure with interconnected pores that facilitate absorption and retention of the wound healing composition. In some cases, gauze may be constructed from cotton, rayon, or synthetic fibers that create a matrix of interwoven strands with varying pore sizes and absorption capacities. The fibrous structure of gauze may allow for deep penetration of the glycerine-based wound healing composition into the material matrix, where the composition may be retained within the fiber interstices and released gradually as the gauze interacts with wound exudate and moisture. The porosity of gauze may facilitate air circulation and moisture vapor transmission, allowing the wound site to maintain appropriate humidity levels while preventing excessive moisture accumulation that could impede healing processes. The flexibility of gauze may enable conformability to wound contours and body movements, where the woven or non-woven structure may accommodate stretching and compression without compromising the integrity of the carrier material or the distribution of the wound healing composition.

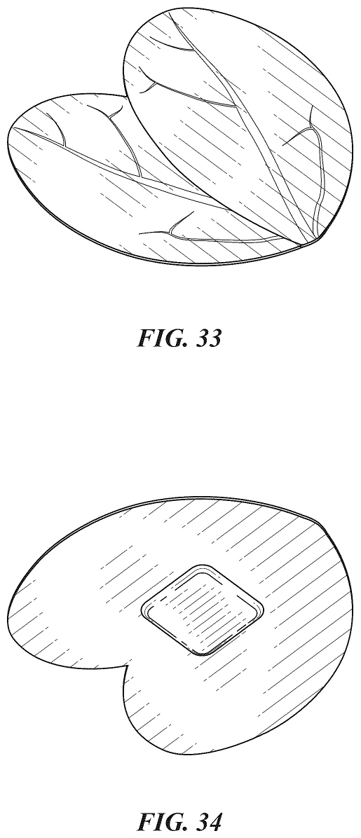

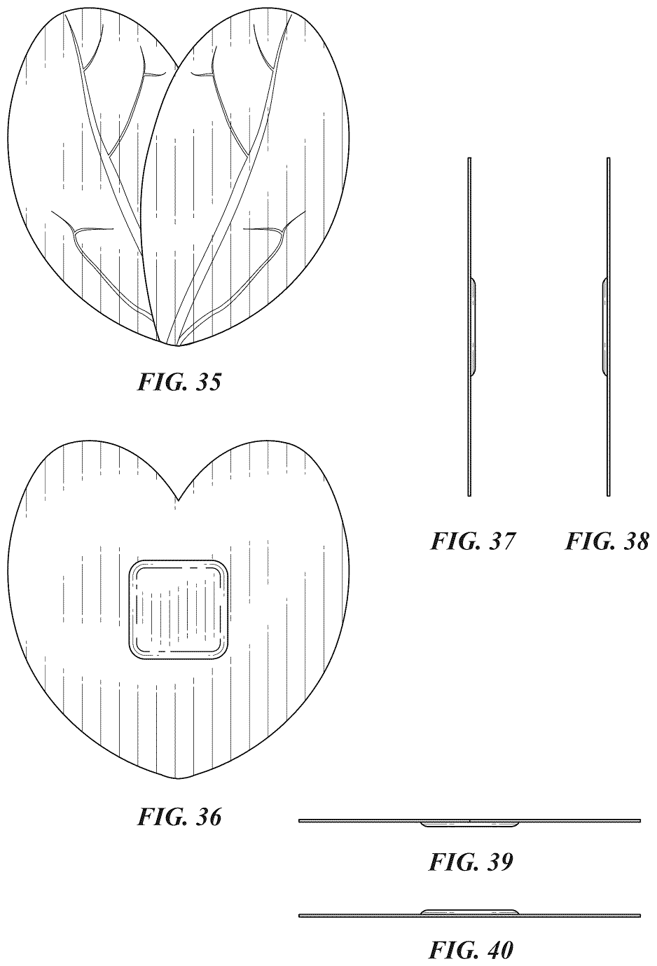

The carrier material may be shaped to resemble lavender (Lavendula angustifolia) petals (FIG. 33) constructed from the combination of two lavender petals including a petal vein pattern extending from a central midrib that distinguishes the lavender-inspired wound dressing from other herbal leaf or petal configurations. In some cases, the carrier material shaped to resemble a heart-shaped combination of lavender petals may comprise a heart-shaped form that provides smaller coverage characteristics suitable for scratches, punctures, or other wounds located in small areas or on small subjects. The smaller heart-shaped geometry may create multiple contact points that distribute adherence forces across smaller surface areas, potentially reducing lengthened pressure concentrations across a larger surface area that could cause discomfort or skin irritation during wear periods. Additional views of the Lavendula angustifolia heart-shaped adhesive wound covering are provided showing a bottom perspective view (FIG. 34), front view (FIG. 35), rear view (FIG. 36), left view (FIG. 37), right view (FIG. 38), top view (FIG. 39), and bottom view (FIG. 40).

Non-woven fabric may serve as an alternative carrier material that provides a fibrous structure created through bonding processes rather than traditional weaving or knitting techniques. In some cases, non-woven fabric may be manufactured using synthetic polymers, natural fibers, or blended materials that are bonded together through thermal, chemical, or mechanical processes to create a cohesive fabric structure. The bonding processes used in non-woven fabric manufacturing may result in a more uniform pore structure compared to woven gauze, where the uniform porosity may provide consistent absorption and retention characteristics for the wound healing composition across the entire surface area of the carrier material. Non-woven fabric may exhibit enhanced strength and dimensional stability compared to traditional gauze, where the bonded fiber structure may resist tearing and deformation during application and wear periods. The manufacturing processes used for non-woven fabric may allow for precise control of material properties such as thickness, density, and porosity, enabling customization of the carrier material characteristics to match specific wound care requirements. The synthetic polymer components commonly used in non-woven fabric may provide chemical resistance and biocompatibility properties that enhance the stability of the wound healing composition and reduce the likelihood of adverse reactions at the wound site.

Hydrocolloid dressing may function as a specialized carrier material that incorporates hydrocolloid particles within a polymer matrix to create a moisture-responsive wound dressing system. In some cases, hydrocolloid dressing may contain gelatin, pectin, carboxymethylcellulose, or other hydrocolloid compounds that absorb wound exudate and form a gel-like layer at the wound interface. The hydrocolloid particles may interact with the glycerine-based wound healing composition to create synergistic moisture management effects, where the hydrocolloid absorption capacity may complement the humectant properties of glycerine to maintain optimal wound hydration levels. The polymer matrix of hydrocolloid dressing may provide structural support and barrier properties that protect the wound site from external contaminants while allowing for controlled moisture exchange. The gel-forming characteristics of hydrocolloid dressing may create a moist wound healing environment that supports cellular migration and tissue regeneration processes, where the gel layer may also provide cushioning effects that reduce mechanical trauma to healing tissue. The adhesive properties of hydrocolloid dressing may eliminate the need for additional securing mechanisms, where the inherent tackiness of the hydrocolloid surface may provide secure adherence to skin surfaces while allowing for atraumatic removal when dressing changes are required.

The wound healing composition may be formulated with varying concentrations of thickening agents based on wound type, where the thickening agent concentration may be adjusted to optimize the viscosity and consistency characteristics for specific wound care applications. In some cases, higher concentrations of thickening agents may be incorporated for highly exudative wounds that produce substantial amounts of wound fluid, where the increased thickening agent content may enhance the absorption capacity and retention properties of the wound healing composition. The higher thickening agent concentrations may create a more viscous gel-like consistency that can absorb and immobilize wound exudate while maintaining the therapeutic properties of the glycerine base and other active ingredients. Xanthan gum may be used at concentrations ranging from 1.5% to 2% by weight for highly exudative wounds, where the increased xanthan gum content may provide enhanced gel strength and fluid absorption capacity. Gellan gum may be incorporated at concentrations of 1% to 2% by weight for highly exudative wound applications, where the higher gellan gum content may create thermoreversible gels with increased viscosity and improved retention characteristics within the carrier material structure.

Lower concentrations of thickening agents may be utilized for dry wounds that produce minimal exudate and require enhanced moisture delivery rather than absorption capacity. In some cases, the reduced thickening agent concentrations may create a more fluid consistency that facilitates penetration of the wound healing composition into dry wound beds and surrounding tissue areas. The lower viscosity formulations may enhance the delivery of glycerine and other moisturizing components to dehydrated wound sites, where the increased fluidity may promote better distribution of therapeutic agents across the wound surface. Xanthan gum may be used at concentrations ranging from 0.5% to 1% by weight for dry wound applications, where the reduced xanthan gum content may provide adequate thickening properties while maintaining sufficient fluidity for enhanced moisture delivery. Gellan gum may be incorporated at concentrations of 0.5% to 1% by weight for dry wound formulations, where the lower gellan gum content may create gels with reduced viscosity that facilitate better penetration and distribution of the wound healing composition. The thickening agent concentration adjustments may be combined with modifications to other formulation components, such as increased glycerine content or enhanced emulsifier concentrations, to create wound healing compositions that are specifically optimized for different wound types and healing stages.

The essential oil selection may include combinations of multiple oils such as chamomile oil, peppermint oil, and eucalyptus oil in addition to tea tree and lavender oil combinations, where the multi-oil formulations may provide synergistic therapeutic effects that address various aspects of wound healing simultaneously. Chamomile oil may contribute anti-inflammatory and soothing properties that complement the antimicrobial effects of tea tree oil and the analgesic properties of lavender oil, where the combination may create a comprehensive therapeutic profile that addresses inflammation, infection risk, and pain management. In some cases, chamomile oil may be incorporated at concentrations ranging from 0.5% to 2% by weight of the wound healing composition, where the chamomile oil content may provide gentle anti-inflammatory effects that are suitable for sensitive skin conditions and irritated wound sites. The bisabolol and chamazulene compounds present in chamomile oil may provide anti-inflammatory activity that helps reduce tissue swelling and inflammatory responses, while the mild sedative properties of chamomile may contribute to pain relief and patient comfort during wound healing processes. Peppermint oil may be added to essential oil combinations to provide cooling sensations and additional antimicrobial properties that enhance the overall therapeutic efficacy of the wound healing composition.

Peppermint oil may be incorporated at concentrations ranging from 0.5% to 1.5% by weight of the wound healing composition, where the menthol content in peppermint oil may provide cooling effects that help reduce pain perception and provide immediate comfort upon application. The antimicrobial properties of peppermint oil may complement the broad-spectrum activity of chelated silver oxide and tea tree oil to create multiple layers of pathogen protection at the wound site. Eucalyptus oil may serve as an additional essential oil component that provides both antimicrobial and anti-inflammatory benefits, where the eucalyptol compound may exhibit activity against various bacterial and fungal pathogens while contributing to the overall therapeutic profile of the multi-oil combination. In some cases, eucalyptus oil may be incorporated at concentrations ranging from 0.5% to 2% by weight of the wound healing composition, where the eucalyptus oil content may provide respiratory benefits through aromatherapeutic effects while delivering topical antimicrobial and anti-inflammatory activity to the wound site. The combination of chamomile oil, peppermint oil, and eucalyptus oil with tea tree oil and lavender oil may create complex essential oil blends that provide comprehensive therapeutic coverage addressing antimicrobial protection, anti-inflammatory activity, pain relief, and sensory comfort aspects of wound care.

The multi-oil essential oil combinations may be formulated to achieve specific therapeutic targets while maintaining compatibility with the glycerine base, emulsifier components, and thickening agents within the wound healing composition. In some cases, the essential oil combinations may be balanced to provide optimal solubility and stability within the glycerine-based matrix, where the emulsifier components such as cetearyl glucoside or phosphatidyl choline may facilitate uniform distribution of the oil-soluble compounds throughout the aqueous glycerine phase. The antimicrobial agent, particularly chelated silver oxide with citric acid as the chelating agent, may work synergistically with the multi-oil essential oil combinations to provide enhanced pathogen protection that addresses both immediate antimicrobial needs and sustained antimicrobial activity throughout the wear period of the wound dressing. The citric acid chelating agent may contribute to the overall stability of the essential oil combinations by providing mild acidic conditions that help preserve the volatile compounds and maintain the therapeutic potency of the oil-based components. The interaction between the essential oil combinations and the carrier material may influence the release characteristics and therapeutic delivery of the wound healing composition, where different carrier materials such as gauze, non-woven fabric, and hydrocolloid dressing may provide varying absorption and retention properties that affect the distribution and availability of the essential oil components at the wound site.

The wound healing composition may include herbal glycerites made from specific medicinal plants including comfrey, Calendula, plantain, lavender, and yarrow as additional active ingredients that enhance the therapeutic profile of the wound dressing system. The herbal glycerites may be prepared using a standardized extraction process that concentrates the bioactive compounds naturally present in each medicinal plant species into a glycerin-based medium that maintains stability and bioavailability of the therapeutic constituents. In some cases, the herbal glycerites may function as concentrated botanical extracts that deliver targeted therapeutic effects based on the specific phytochemical profiles of the individual plant materials. The comfrey glycerite may provide allantoin and mucilage compounds that support tissue regeneration and cellular repair processes, while the Calendula glycerite may contribute triterpenoids and polyphenols that offer anti-inflammatory and antimicrobial benefits. The plantain glycerite may deliver polysaccharides and flavonoids that provide wound healing and anti-inflammatory activity, whereas the lavender glycerite may supply linalool and other aromatic compounds that contribute analgesic and antimicrobial properties. The yarrow glycerite may contain achilleine and other bioactive constituents that provide astringent and hemostatic effects that help control bleeding and reduce exudate production at wound sites.

The glycerite components may be prepared using a specific process involving a 75% glycerin and 25% distilled water ratio, with a 3:1 liquid to herb ratio for dried herbs, heated gently in a double boiler for at least 40 minutes to extract the bioactive compounds from the plant material into the glycerin-water mixture. The 75% glycerin concentration may provide optimal extraction efficiency for both water-soluble and lipophilic compounds present in the medicinal plant materials, where the glycerin component may function as a universal solvent that can extract a broad spectrum of therapeutic constituents. The 25% distilled water component may enhance the extraction of water-soluble compounds such as polysaccharides, glycosides, and phenolic acids that contribute to the wound healing properties of the herbal materials. In some cases, the 3:1 liquid to herb ratio may ensure adequate solvent volume to fully saturate the dried plant material and facilitate complete extraction of the available bioactive compounds. The dried herb preparation may involve grinding or chopping the plant material to increase surface area and improve extraction efficiency, where the particle size reduction may allow for better penetration of the glycerin-water mixture into the cellular structures of the plant material.

The gentle heating process in a double boiler for at least 40 minutes may facilitate the extraction of heat-stable compounds while preventing thermal degradation of temperature-sensitive bioactive constituents. The double boiler heating method may provide controlled temperature conditions that maintain the extraction temperature below the boiling point of water, where the indirect heating may prevent overheating that could damage volatile compounds or cause caramelization of the glycerin component. In some cases, the heating duration of at least 40 minutes may allow sufficient time for the glycerin-water mixture to penetrate the plant cell walls and dissolve the intracellular compounds into the extraction medium. The extraction temperature may be maintained between 60 °C and 80 °C during the heating process, where the elevated temperature may enhance the solubility of the bioactive compounds while preserving the integrity of heat-sensitive constituents. The gentle heating conditions may also facilitate the breakdown of cellular structures within the plant material, allowing for more complete extraction of compounds that may be bound within cell walls or other structural components. Following the heating period, the plant material may be separated from the liquid extract through filtration or straining processes that remove the solid residue while retaining the glycerite solution containing the concentrated bioactive compounds.

The wound healing composition may include hypericum extract, also known as St. John's wort extract, containing hypericin, hyperforin, and other active compounds as an additional ingredient that provides antimicrobial and wound healing properties to the formulation. Hypericum perforatum may serve as the source material for the hypericum extract, where the flowering tops and leaves of the plant may contain the highest concentrations of the therapeutically active compounds. Hypericin may function as a naphthodianthrone compound that exhibits antimicrobial activity against gram-positive bacteria and antiviral properties that help prevent secondary infections at wound sites. In some cases, hypericin may also provide photodynamic effects that enhance antimicrobial activity when exposed to light, where the photosensitizing properties may contribute to pathogen elimination in wound environments. Hyperforin may serve as a phloroglucinol derivative that demonstrates broad-spectrum antimicrobial activity against various bacterial strains, including some antibiotic-resistant organisms that may be encountered in chronic wound conditions. The hyperforin component may also exhibit anti-inflammatory properties that help reduce tissue swelling and inflammatory responses during wound healing processes.

The hypericum extract may be prepared as a hypericum glycerite using the same standardized extraction process involving a 75% glycerin and 25% distilled water ratio, with a 3:1 liquid to herb ratio for dried hypericum material, heated gently in a double boiler for at least 40 minutes. The hypericum glycerite preparation may concentrate the hypericin and hyperforin compounds along with other bioactive constituents such as flavonoids, tannins, and essential oils that contribute to the overall therapeutic profile of the extract. Flavonoids present in hypericum may include quercetin, rutin, and hyperoside, where these compounds may provide antioxidant and anti-inflammatory effects that complement the antimicrobial activity of hypericin and hyperforin. The tannin components in hypericum may contribute astringent properties that help tighten tissues and reduce bleeding at wound sites, while the essential oil constituents may provide additional antimicrobial and aromatic properties. In some cases, the hypericum glycerite may be incorporated into the wound healing composition at concentrations ranging from 1% to 10% by weight, where the hypericum content may be adjusted based on the desired antimicrobial potency and the specific wound care application requirements.

The wound healing composition may include seaweed-derived compounds such as alginate, laminaran, and other bioactive compounds from kelp and kombu as additional active ingredients that provide moisture management and wound healing enhancement properties. Alginate may function as a highly absorbent polysaccharide derived from brown seaweed species, where the alginate component may absorb up to 20 times its weight in fluid while forming a gel-like matrix that maintains moisture balance at the wound site. The alginate absorption capacity may complement the humectant properties of glycerine to create synergistic moisture management effects that support optimal wound healing conditions. In some cases, alginate may be incorporated as sodium alginate or calcium alginate, where the ionic form may influence the gel-forming characteristics and absorption properties of the compound within the wound healing composition. The gel-forming properties of alginate may create a protective barrier over the wound surface that helps prevent bacterial contamination while allowing for controlled moisture exchange between the wound bed and the surrounding environment.

Laminaran may serve as a polysaccharide compound found in brown seaweeds that provides anti-inflammatory, antioxidant, antiviral, anticoagulant, and antitumoral properties that enhance the therapeutic profile of the wound healing composition. The anti-inflammatory properties of laminaran may help reduce tissue swelling and inflammatory responses that could impede wound healing progress, where the anti-inflammatory activity may complement the effects of other anti-inflammatory components such as essential oils and herbal glycerites. The antioxidant properties of laminaran may protect wound tissue from oxidative stress and free radical damage during the healing process, where the antioxidant effects may help preserve cellular integrity and support optimal conditions for tissue regeneration. In some cases, the antiviral properties of laminaran may provide protection against viral pathogens that could potentially compromise wound healing, while the anticoagulant properties may help maintain appropriate blood flow and prevent excessive clot formation that could impede healing processes. The antitumoral properties of laminaran may contribute to cellular regulation effects that support normal tissue growth and prevent abnormal cellular proliferation during wound healing and tissue remodeling phases.

Kelp and kombu may serve as sources of additional bioactive compounds that can be incorporated into the wound healing composition through fermentation processes that release active substances including polysaccharides, fucose, and alginic acid. The fermentation process may involve controlled microbial activity that breaks down the cellular structures of the seaweed materials and releases bioactive compounds that may not be readily available through conventional extraction methods. Polysaccharides derived from kelp and kombu through fermentation may include fucoidan and other sulfated polysaccharides that provide anti-inflammatory and immunomodulatory effects that support wound healing processes. Fucose may function as a monosaccharide component that contributes to the biological activity of the seaweed-derived polysaccharides, where the fucose content may influence the therapeutic potency and bioavailability of the seaweed extracts. Alginic acid may serve as the acidic form of alginate that provides similar moisture management properties while contributing to the overall stability and pH balance of the wound healing composition.

The seaweed-derived compounds may be incorporated into the wound healing composition at varying concentrations based on the desired therapeutic effects and the specific wound care application requirements. In some cases, alginate may be included at concentrations ranging from 0.5% to 5% by weight of the wound healing composition, where the alginate content may be adjusted to provide appropriate absorption capacity for different wound exudate levels. Laminaran may be incorporated at concentrations ranging from 0.1% to 2% by weight, where the laminaran content may be optimized to provide anti-inflammatory and antioxidant benefits without compromising the stability or consistency of the formulation. The fermented kelp and kombu extracts may be added at concentrations ranging from 0.5% to 3% by weight, where the extract content may be balanced to provide bioactive compound delivery while maintaining compatibility with other formulation components. The combination of alginate, laminaran, and fermented seaweed extracts may create synergistic effects that enhance both the moisture management and therapeutic properties of the wound healing composition, where the seaweed-derived compounds may work in conjunction with the glycerine base, essential oils, and herbal glycerites to provide comprehensive wound care benefits.

A method of treating a wound may comprise applying to a wound a wound dressing comprising a carrier material infused with a wound healing composition, where the application process may involve selecting appropriate leaf-shaped designs based on specific wound characteristics and therapeutic requirements. The wound healing composition may comprise glycerine as a base component that provides humectant properties for maintaining optimal moisture levels at the wound site. In some cases, the glycerine component may work in conjunction with at least one emulsifier to create a stable formulation that facilitates uniform distribution of active ingredients throughout the carrier material structure. The at least one thickening agent incorporated into the wound healing composition may provide appropriate viscosity characteristics that enhance retention of the therapeutic agents within the carrier material while allowing for controlled release during the treatment period. The carrier material may be shaped to resemble a leaf of a plant known for wound-healing properties, where the leaf-inspired design may provide both aesthetic appeal and functional benefits that enhance patient compliance and treatment outcomes.

The selection of appropriate leaf shapes for different wound types may be based on the specific therapeutic properties associated with different medicinal plant species and the geometric characteristics that optimize coverage and adherence for particular wound configurations. In some cases, wounds located on linear body surfaces or surgical incision sites may benefit from elongated leaf shapes such as plantain or Arnica leaf designs that provide directional coverage along the wound axis. The plantain leaf-shaped carrier material may be selected for wounds that require comprehensive antimicrobial protection, where the elongated form with tapered ends and central vein structure may facilitate application over wounds that extend across body contours. Wounds with irregular geometries or those requiring enhanced tissue regeneration support may benefit from broader leaf shapes such as comfrey leaf designs that provide increased surface area coverage. The comfrey leaf-shaped carrier material with its broader, oval shape and mosaic pattern may be particularly suitable for wounds that require sustained delivery of regenerative compounds and comprehensive surface protection.

Circular or oval wounds may be treated using Calendula leaf-shaped carrier materials that incorporate serrated edges and rounded overall forms that conform to curved wound geometries. The Calendula leaf design may be selected for wounds that require continuous monitoring throughout the healing process, where the display window feature incorporated into the central region allows for visual inspection of wound healing progress without disrupting the therapeutic environment through premature dressing removal. Wounds associated with significant pain or discomfort may benefit from Arnica leaf-shaped carrier materials that provide enhanced analgesic properties through the elongated oval form and leaf vein pattern extending from a central midrib. The Arnica leaf design may facilitate targeted delivery of pain-relieving compounds to wound sites while providing structural reinforcement through the vein pattern that maintains carrier material integrity during wear periods.

The wound healing composition infused into the selected carrier material may comprise at least one essential oil that provides therapeutic benefits tailored to specific wound care requirements and healing stages. Tea tree oil and lavender oil may be incorporated as the at least one essential oil to create synergistic effects that address multiple aspects of wound healing simultaneously. In some cases, the wound healing composition may comprise tea tree oil and lavender oil as the at least one essential oil, where the tea tree oil component may provide broad-spectrum antimicrobial activity against bacterial pathogens that could compromise wound healing processes. The antimicrobial properties of tea tree oil may help prevent bacterial colonization at the wound site while supporting the maintenance of a clean wound environment conducive to natural healing processes. The lavender oil component may contribute anti-inflammatory and analgesic properties that reduce pain perception and tissue swelling associated with wound healing, where the combination of tea tree oil and lavender oil may create complementary therapeutic effects that enhance both pathogen protection and patient comfort during treatment.

The antimicrobial protection provided by the wound healing composition may be enhanced through the incorporation of an antimicrobial agent that works synergistically with the essential oil components to provide comprehensive pathogen control. The antimicrobial agent may comprise chelated silver oxide with citric acid as a chelating agent, where the chelation process may improve the stability and bioavailability of the silver component within the glycerine-based formulation. In some cases, the chelated silver oxide may be present in an amount of 0.1-5% by weight of the wound healing composition, where the concentration range may be adjusted based on the antimicrobial requirements of specific wound types and the desired duration of antimicrobial activity. Lower concentrations within the 0.1-1% range may be suitable for minor wounds or wounds with low infection risk, while higher concentrations within the 2-5% range may be appropriate for wounds with elevated infection potential or chronic wounds that require sustained antimicrobial protection.

The citric acid chelating agent may enhance the solubility and stability of the silver oxide component while contributing mild acidic properties that help maintain optimal pH conditions for wound healing processes. The chelated silver oxide may provide sustained antimicrobial activity throughout the wear period of the wound dressing, where the controlled release of silver ions may create a protective antimicrobial barrier that prevents pathogen proliferation without causing cytotoxic effects to healthy tissue. The combination of chelated silver oxide with essential oils may create multiple layers of antimicrobial protection that address different pathogen types and resistance mechanisms, where the multi-modal antimicrobial approach may reduce the likelihood of resistance development and provide comprehensive pathogen control across various wound environments.

The application method may involve preparing the wound site through appropriate cleansing and debridement procedures before applying the selected herbal-shaped wound dressing to ensure optimal contact between the wound healing composition and the wound bed. This could also include the ability for transdermal delivery of the wound healing composition. The carrier material infused with the wound healing composition may be positioned over the wound site with consideration for the directional characteristics of the leaf shape and the anatomical features of the treatment area. In some cases, the central vein or midrib regions of leaf-shaped carrier materials may be aligned with the primary axis of linear wounds to optimize the distribution of therapeutic agents along the wound length. The irregular edges and natural contours of the leaf-shaped designs may be positioned to conform to the wound margins while providing adequate coverage of surrounding healthy tissue to prevent edge contamination and support adherence stability.

The therapeutic benefits achieved through the combination of herbal compositions and leaf-inspired designs may include enhanced moisture management, sustained antimicrobial protection, anti-inflammatory activity, and improved patient acceptance of wound care regimens. The glycerine base component of the wound healing composition may provide humectant effects that maintain optimal hydration levels at the wound site, where the moisture retention properties may support cellular migration and tissue regeneration processes throughout the healing phases. The at least one emulsifier may facilitate uniform distribution of the essential oil components and antimicrobial agents throughout the carrier material structure, ensuring consistent therapeutic agent delivery across the entire wound surface. The at least one thickening agent may create appropriate viscosity characteristics that enhance the retention of the wound healing composition within the carrier material while allowing for controlled release of active ingredients as the dressing interacts with wound exudate and physiological moisture.

The leaf-inspired designs may provide biomimetic characteristics that enhance the conformability and adherence properties of the wound dressing while creating visual identifiers that facilitate appropriate product selection for different wound care applications. The natural contours and edge patterns of the leaf shapes may distribute mechanical stress more evenly across the wound site compared to geometric dressing designs, where the organic shapes may accommodate body movements and wound site dynamics without compromising dressing integrity or therapeutic agent distribution. The combination of shape and composition may work synergistically to create wound care solutions that address both the physical and biochemical requirements for optimal healing outcomes, where the herbal-inspired designs may enhance patient engagement and compliance with treatment protocols through improved aesthetic appeal and functional performance. The method of treatment application may result in comprehensive wound care that leverages the therapeutic properties of natural compounds with the practical advantages of biomimetic design principles to support effective wound healing across various wound types and patient populations.

A number of implementations have been described. Nevertheless, it will be understood that various modifications may be made without departing from the spirit and scope of the disclosure. Accordingly, other implementations are within the scope of the following claims.

I claim:

1. A wound healing composition, comprising:
glycerine;
at least one emulsifier selected from any one or more of a group consisting of cetearyl glucoside, cetearyl alcohol, and phosphatidyl choline;
at least one or more thickening agent selected from a group consisting of xanthan gum and gellan gum;
at least one or more essential oils selected from a group consisting of tea tree oil, lavender oil, helichrysum oil, frankincense oil, menthol oil, peppermint oil, lavender oil, eucalyptus oil, chamomile oil, rose oil, geranium oil, and neroli oil, tea tree oil, lavender oil, chamomile oil, and peppermint oil; and
chelated silver oxide, wherein said chelating agent is citric acid.

2. The wound healing composition of claim 1, wherein the glycerine is present in an amount of 60-70% by weight of the composition.

3. The wound healing composition of claim 1, wherein the at least one emulsifier is present in an amount of 5-15% by weight of the composition.

4. The wound healing composition of claim 3, wherein the at least one emulsifier is cetearyl glucoside.

5. The wound healing composition of claim 1, wherein the at least one thickening agent is present in an amount of 0.5-2% by weight of the composition.

6. The wound healing composition of claim 1, wherein the at least one essential oil is present in an amount of 1-5% by weight of the composition.

7. The wound healing composition of claim 6, wherein the at least one essential oil comprises a combination of tea tree oil and lavender oil.

8. The wound healing composition of claim 1, wherein the chelated silver oxide is present in an amount of 0.1-5% by weight of the composition.

9. A wound dressing, comprising:
a carrier material shaped to resemble a petal or a leaf of a plant known for wound-healing properties; and
a wound healing composition infused into the carrier material, the wound healing composition comprising glycerine, at least one emulsifier, at least one thickening agent, at least one essential oil, and an antimicrobial agent.

10. The wound dressing of claim 9, wherein the carrier material is selected from the group consisting of gauze, non-woven fabric, and hydrocolloid dressing.

11. The wound dressing of claim 9, wherein the plant known for wound-healing properties is selected from the group consisting of narrow leaf plantain (Plantago lanceolata), yarrow (Achillea millefolium), broadleaf plantain (Plantago major), comfrey (Symphytum officinale), chamomile (Chamomilla recutita), Calendula (Calendula officinalis), Arnica (Arnica montana), marsh mallow (Althaea officinalis L. (Malvaceae), St. John's Wort (Hypericum perforatum), lavender (Lavendula angustifolia). comfrey (Symphytum officinale), and aloe vera (Aloe barbadensis) plantain (Plantago lanceolata), comfrey (Symphytum officinale), Calendula (Calendula officinalis), yarrow (Achillea millefolium), and lavender (Lavandula).

12. The wound dressing of claim 11, wherein the carrier material is shaped to resemble a plantain leaf comprising an elongated form with tapered ends and a central vein structure.

13. The wound dressing of claim 11, wherein the carrier material is shaped to resemble a comfrey leaf.

14. The wound dressing of claim 11, wherein the carrier material is shaped to resemble Calendula leaf.

15. The wound dressing of claim 11, wherein the carrier material is shaped to resemble a comfrey leaf comprising a broader, oval shape with a mosaic pattern across its surface.

16. The wound dressing of claim 9, wherein the antimicrobial agent comprises chelated silver oxide with citric acid as a chelating agent and wherein the dressing is also a covering.

17. A method of treating skin irritations, comprising:
applying to a wound, abrasion, cut or scrape a dressing comprising a carrier material infused with a wound healing composition, wherein the dressing composition comprises glycerine, at least one emulsifier, at least one thickening agent, at least one essential oil, and an antimicrobial agent, and wherein the carrier material is shaped to resemble a petal or a leaf of a plant known for wound-healing properties.

18. The method of claim 17, wherein the plant known for wound-healing properties is selected from the group consisting of plantain (Plantago major), comfrey (Symphytum officinale), Calendula (Calendula officinalis), yarrow (Achillea millefolium), and lavender (Lavandula).

19. The method of claim 16, wherein the carrier material is shaped to resemble a plantain leaf comprising an elongated form with tapered ends and a central vein structure.

20. The method of claim 18, wherein the carrier material is shaped to resemble a Calendula leaf comprising serrated edges and a rounded overall form with a display window incorporated into a central region, wherein the display window allows for visual inspection of the covered wound area without removing the wound dressing.

21. The method of claim 17, wherein the wound healing composition comprises tea tree oil and lavender oil as the at least one essential oil.

22. The method of claim 17 wherein the antimicrobial agent comprises chelated silver oxide with citric acid as a chelating agent, and wherein the chelated silver oxide is present in an amount of 0.1-5% by weight of the wound healing composition.
